SEQUENCE LISTING (18 sequences)

SEQ ID NO: 1 - DNA, 84 bp, Artificial Sequence
  Synthetic polynucleotide
gatcgtaccaattggagcttaggagatgacacttctattcaagagatagaagtgtcatctcctaagctttttttcaattggtac

SEQ ID NO: 2 - DNA, 84 bp, Artificial Sequence
  Synthetic polynucleotide
ttaagtaccaattgaaaaaaagcttaggagatgacacttctatctcttgaatagaagtgtcatctcctaagctccaattggtac

SEQ ID NO: 3 - DNA, 84 bp, Artificial Sequence
  Synthetic polynucleotide
gatcgtaccaattggtctgtcaagttcatcaagagtgttcaagagacactcttgatgaacttgacagattttttcaattggtac

SEQ ID NO: 4 - DNA, 84 bp, Artificial Sequence
  Synthetic polynucleotide
ttaagtaccaattgaaaaaatctgtcaagttcatcaagagtgtctcttgaacactcttgatgaacttgacagaccaattggtac

SEQ ID NO: 5 - DNA, 84 bp, Artificial Sequence
  Synthetic polynucleotide
gatcgtaccaattggaacctagaggatgaagatggaattcaagagattccatcttcatcctctaggttttttttcaattggtac

SEQ ID NO: 6 - DNA, 84 bp, Artificial Sequence
  Synthetic polynucleotide
ttaagtaccaattgaaaaaaaacctagaggatgaagatggaatctcttgaattccatcttcatcctctaggttccaattggtac

SEQ ID NO: 7 - DNA, 84 bp, Artificial Sequence
  Synthetic polynucleotide
gatcgtaccaattggccttgagaatcattctaagatcctcaagagggatcttagaatgattctcaaggttttttcaattggtac

SEQ ID NO: 8 - DNA, 84 bp, Artificial Sequence
  Synthetic polynucleotide
ttaagtaccaattgaaaaaaccttgagaatcattctaagatccctcttgaggatcttagaatgattctcaaggccaattggtac

SEQ ID NO: 9 - DNA, 20 bp, Artificial Sequence
  Synthetic polynucleotide
gccccatggctgactaattt

SEQ ID NO: 10 - DNA, 20 bp, Artificial Sequence
  Synthetic polynucleotide
gtatcgccctcgaacttcac

SEQ ID NO: 11 - DNA, 19 bp, Artificial Sequence
  Synthetic polynucleotide
agcaggacctaggcctgct

SEQ ID NO: 12 - DNA, 20 bp, Artificial Sequence
  Synthetic polynucleotide
cttcttgctcccatcttgcc

SEQ ID NO: 13 - DNA, 20 bp, Artificial Sequence
  Synthetic polynucleotide
agcctcctacaacccaacct

SEQ ID NO: 14 - DNA, 20 bp, Artificial Sequence
  Synthetic polynucleotide
ctgtcgctccagcttttctg

SEQ ID NO: 15 - DNA, 20 bp, Artificial Sequence
  Synthetic polynucleotide
gcctggtagccacactccaa

SEQ ID NO: 16 - DNA, 20 bp, Artificial Sequence
  Synthetic polynucleotide
aagctcagagcaaggctcca

SEQ ID NO: 17 - DNA, 20 bp, Artificial Sequence
  Synthetic polynucleotide
cgacgggaatatgtcctacg

SEQ ID NO: 18 - DNA, 20 bp, Artificial Sequence
  Synthetic polynucleotide
attccatcctcgaactgccc

(19) United States
(12) Patent Application Publication
(10) Pub. No.: US 2012/0017289 A1
(43) Pub. Date: Jan. 19, 2012

(54) RAPID METHOD FOR GENERATING GENE KNOCK DOWN MODEL

(75) Inventors: Subeer Suhash Majumdar, New Delhi (IN); Deepika Sharma, New Delhi (IN); Neerja Wadhwa, New Delhi (IN)

(73) Assignee: National Institute of Immunology, New Delhi (IN)

(21) Appl. No.: 12/895,913

(22) Filed: Oct. 1, 2010

(30) Foreign Application Priority Data
Jul. 14, 2010 (IN) 1649/DEL/2010

(51) Int. Cl. C12N 15/85

(52) U.S. Cl. 800/21

(57) ABSTRACT

The present invention provides a novel method of generating knock down models by electroporation of shRNA construct into the testis. The present invention provides an ethically superior, non-surgical, user friendly rapid method for the generation of permanent lines of shRNA knock down non human vertebrates. This invention is ethically superior as it does not involve any loss of animal life and drastically minimizes the production time and use of animals. Current techniques for making knockout models are cumbersome, require trained personnel, costly infrastructure and require hundreds of eggs collected after killing several females. In contrast, this method neither involves any costly infrastructure nor requires trained personnel. The invention also relates to the quick incorporation of shRNA gene construct into the germline of a species so that shRNA is inheritable. The present invention also generates in a single go a variety of knock down models differentially expressing gene specific shRNA, depending on differential shRNA gene incorporation in native genome of various male germ cells, so that there is no restriction in the choice of the gene knock down.

### FIELD OF INVENTION

The present invention is directed towards improvement in the method of producing knock down models using shRNA examples not limited to guinea pig, rabbit, rat, mice, dog, etc. The present invention also involves making permanent lines of shRNA knock down models produced by the method. A novel, ethically superior, non surgical, user friendly, less time-consuming and relatively inexpensive rapid method has been invented for making permanent lines of non human vertebrates. A specific gene is knocked down by electroporating shRNA construct into the testis of a non human vertebrate. The present invention does not involve any loss of life. The invention also relates to the incorporation of genes into the germline of non human vertebrate so that it could be inherited. The method is the easiest and fastest in comparison to all other existing methods.

### BACKGROUND OF THE INVENTION

A gene is important for the vital functioning of the organism. A gene's biological role in an organism can only be fully appreciated by observing the phenotypic consequences of altering its function. Transgenic technology, which allows the insertion of exogenous genes or the alteration or disruption of endogenous genes, has emerged as a powerful tool for the analysis of gene function. Since the publication of the first transgenic model in 1980, great efforts have been implemented to improve the efficiency of these techniques, but still the manipulation of genes remains difficult and time-consuming. The generation of a knock out mouse requires years of work and sometimes does not yield desired results. This accounts for one of the main reasons why the published knock outs represent only 10% of the 30,000 mouse genes. With the availability of complete human and mouse genomes, use of knock out models to study gene function is bound to increase.

The standard approach for loss of function studies is to generate a knock out model in which the gene of interest is inactivated through homologous recombination in embryonic stem (ES) cells. Although this approach is very powerful, it suffers from major drawbacks. The frequency of homologous recombination per electroporated cell, is approximately 10⁻⁵ to 10⁻⁶. The efficiency of homologus recombination is extremely poor and this procedure is very time consuming. A knock out construct making involves many steps of molecular manipulations. Further, after the construct is made, there are still months of time required for ES cell culturing, clone identification, chimera production, and breeding for germ line transmission. The other drawback of the knockout approach is that the so produced mutant allele is null (resulting into a complete loss of function). Although the total loss of function of a gene is very powerful in revealing its overall function, it is not always the most informative, especially when it causes early embryonic lethality. Hence, graded loss of function may provide more information about a gene than a complete loss. Kuhn et al. (2009) also provided a complicated method of shRNA mediated conditional knock down of genes by integrating the combination of DNA molecule into the genome of a mammal and crossing the said mammal with a mammal transgenic for an expressible recombinase gene.

There are few surgical methods also available where testis has been used for overexpression of the gene. Shinohara and Shinohara (2008) described a method for the introduction of gene in spermatogenic cell, where the desired gene was overexpressed in the testis. Using surgical method, they injected the gene into the seminiferous tubules of an immature testis through efferent duct to generate a transgenic model. Nakatsuji et al. (2002) also described a surgical method for production of a transgenic model by overexpressing a foreign gene into the testis at the age of 14 days.

With the advent of RNA interference (RNAi) technology, it has become very easy to specifically knock down the function of a particular gene of interest. A function of gene can be studied efficiently by inhibiting its action by RNAi, thereby making this technology popular in the era of human genome for investigating the function of newly identified genes. Recently, many laboratories have started using RNAi technology to knock down gene expression. RNAi is the process by which double stranded RNA silences gene expression, either by inducing the sequence-specific degradation of complementary mRNA or by inhibiting translation. This leads to a decrease (but not absence) of expression of the corresponding protein. After more than two decades of functional research aimed at developing and continuously improving transgenic and knock out technology, the advent of RNAi knock-down represents a valuable new alternative for studying gene function. The discovery of small interfering RNA duplexes (siRNA) which silences the gene expression in mammalian cells has revolutionized biomedical research. However, the knock down effect of siRNA is only transient. Persengiev et al. (2004) reported that the effect of siRNA is sometimes non specific. To achieve a more sustained and specific gene silencing effect, small hairpin RNA (shRNA) expressed from a vector is preferred. With the advent of shRNA system, the mechanism of RNAi has been used for tissue specific knock down of genes promising to provide a quicker and cheaper way to generate knock down models than conventional approaches.

For shRNA to be widely accepted as a gene silencing tool in mammals, significant technical challenges will need to be overcome. These challenges include developing efficient ways of designing, identifying and delivering effective shRNAs. To increase the transfection efficiency in culture system, the delivery has been improved by the use of lipid mediated system and by the use of various adeno-, lenti- or retro-viruses. However, these approaches are limited by the transient nature of the response and in some cases by lipid-mediated toxicity. Apart from these limitations, there are certain other limitations of biological processes involving genes function that cannot be recapitulated in cultured cells because biological membrane barriers are absent in a petri dish. Therefore, it is desirable to apply RNAi technology in the mammalian system. Until now, few studies have reported successful systemic shRNA delivery in certain models, and strategies to achieve this are highly sought thereafter. The available techniques of delivering shRNA have not paved an easier way for germ line transmission as existing methodologies suffer from several constraints and loss of time. In one of these methods, where ES cell clones with RNAi effect were generated and then used to produce the knock out model, the method was almost as time consuming as the conventional knockout. The second method of infecting or injecting one cell zygotes with lentiviruses carrying a shRNA expressing cassette requires the production of high titer lentiviruses is laborious and technically challenging. Introducing shRNA expressing constructs as transgenes via conventional pronuclear microinjection in one cell zygotes is labor intensive, time consuming and requires hundreds of zygotes collected from several females upon superovulation and has limited success rate of the procedure. Hence, delivery of shRNA to ensure appropriate incorporation is a daunting barrier to successful RNAi technology.

According to Hirabayashi et al (2001) pronuclear microinjection method for generating transgenic models suffers from very low efficiency ranging from 0.5% to 10%. Several attempts have been made to improve the efficiency of genomic integration of foreign DNA. Page et al. (1995) attempted to increase the efficiency by cytoplasmic injection of DNA mixed with polylysine. Seo et al. (2000) doubled the efficiency of transgenesis by co-injecting restriction endonuclease together with foreign DNA into mouse pronuclei. Hirabayashi et al. (2001) used zygote centrifugation to visualize pronuclei and generate transgenics. Nottle et al. (2001) also tested the effect of DNA concentration on the rate of transgenesis.

All these generates an imperative requirement of an easily adoptable rapid approach for obtaining desired knockout model, preferably with more stability and less complexity for general use. The transgenic RNAi generated may serve as a “hypomorphic allele” and not as a null mutant where the expression of endogenous genes is not completely repressed. It may be used as an approach to study functions of genes, especially to those whose absence results in embryonic or early postnatal lethality. Generation of knock down model carrying mild phenotypes or various hypomorphic alleles could greatly facilitate studies in many fields of biological sciences. We believe that the scope of shRNA work will be vast if one can generate several transgenic lines with different degrees of loss of expression/function for a single gene, thus creating many hypomorphic alleles. Exploiting RNAi by overexpression of shRNA in transgenic models is a quicker and more versatile approach compared to the knock out models in the study of gene function.

With great efforts one may obtain a knock out model using pronuclear microinjection, but many a times one may not get the desired level of mRNA degradation necessitating more labour and more deaths for generation of more transgenic lines to achieve satisfactory outcome. Therefore, it is necessary to develop a more comprehensible approach which can also provide opportunity to generate variety of models expressing shRNA differentially so that the choice of extent of desired shRNA expression also becomes available in one go with minimal use of mammals.

In view of the above described circumstances, a non-surgical, user friendly, less time consuming, more effective and relatively inexpensive technique is needed to generate knock down models. The present invention provides a method which is an electroporation mediated method, which does not involve any loss of non human vertebrate life as required in other available techniques. Minimization of the number of animals used and their suffering is probably one of the most important issues in biological research. Present invention has addressed these issues successfully by developing a non invasive method for making permanent lines of non human vertebrates where a specific gene is knocked down by shRNA.

### OBJECTIVE OF THE INVENTION

Accordingly, the main objective of this invention is to provide a process for generating shRNA knock down model, which is non-surgical, user friendly, less time consuming, more effective and relatively inexpensive.

Yet another objective of this invention is to provide a process for generation of shRNA knock down model for all non human vertebrates and to make permanent lines of shRNA knock down models.

### STATEMENT OF INVENTION

Accordingly, the present invention relates to a process that is non-surgical, user friendly, less time consuming, more effective and relatively inexpensive to generate shRNA knock down model(s). By this process, various shRNA knock down models are generated for different genes illustratively Interferon stimulated gene 12, Transglutaminase 2, Nuclear protein 1 and Glioma tumour suppressor candidate region gene 2. The present process generates in a single batch, a variety of knock down models differentially expressing gene specific shRNA depending on differential shRNA incorporation in native genome, so that the choice of the extent of gene knock down becomes available in a single process.

### BRIEF DESCRIPTION OF DRAWINGS

FIG. 1 shows a diagram of linearised shRNA vector consisting of CMV promoter which drives the expression of shRNA and a SV40 promoter which drives the expression of the GFP. Gene specific shRNA sequences can be easily inserted into the vector in between BamH1 and AfIII sites. This vector carries GFP for convenient tracking.

FIG. 2: shows the PCR results of genomic DNA (gDNA) of F1 progeny generated from the mating of electroporated male and wild type female.
Lane 1-18 represents PCR of ISG12 (Interferon stimulated gene 12)
Lane 1: gDNA of wild type, Lane 2-16: gDNA of ISG12, Lane 17: plasmid DNA,
Lane 18: 100 bp marker
Lane 19-32 represents PCR of TGM2 (Transglutaminase 2)
Lane 19: gDNA of wild type, Lane 20-30: gDNA of TGM2, Lane 31: plasmid DNA,
Lane 32: 100 bp marker
Lane 33-49 represents PCR of NUPR1 (Nuclear protein 1)
Lane 33-47: gDNA of NUPR1 mice, Lane 48: gDNA of wild type, Lane 49: plasmid DNA
Lane 50-69 represents PCR of GLTSCR2 (Glioma tumour suppressor candidate region gene 2)
Lane 50-66: gDNA of GLTSCR2 mice, Lane 67: gDNA of wild type, Lane 68: plasmid DNA, Lane 69: 100 bp marker.

FIG. 3: shows the GFP expression in the whole testes of ISG12 electroporated model.
A) Phase contrast image of the testis of ISG12 electroporated model.
B) Fluorescent image of the testis of ISG12 electroporated model showing green GFP expression.

FIG. 4: shows the immunohistochemical localization of GFP expression in the testes of shRNA knock down model. GFP antibody staining of bouin's fixed, paraffin embedded testicular sections. Note the green GFP staining. A) and B) Fluorescence and merged image of the testicular sections of Wild Type. C) and D) Fluorescence and merged image of the testicular sections of ISG12 model. E) and F) Fluorescence and merged image of the testicular sections of TGM2 model.

FIG. 5: shows the immunolocalization of TGM2 protein in testicular sections of TGM2 shRNA knock down model and wild type. A) and B): Fluorescence and merged image of the seminiferous tubules of wild type model. C) and D) Fluorescence and merged image of the seminiferous tubules of TGM2 shRNA knock down model. Very few cells stained positive for TGM2.

FIG. 6: shows the down regulation of mRNA levels in testes of shRNA knock down model relative to Wild type testes at ten weeks of age. Number of asterisks represents degree of statistical significance (P<0.05). (n=3)

FIG. 7: shows the western blot analysis of TGM2 protein levels from the testes of TGM2 shRNA knock down model and wild type. Lane 1-3: Testes from three different TGM2 shRNA knock down models. Lane 4-6: Testes from three different wild types.

FIG. 8: A) shows the graphical representation of Mean Testis Weight (in mgs) of Wild Type and shRNA knock down models at ten weeks of age. Number of asterisks represents degree of statistical significance (P<0.05) (n=10).
B) shows the graphical representation of mean sperm count (million/ml) from epididymis of wild type and shRNA knock down model at ten weeks of age. Number of asterisks represents degree of statistical significance (P<0.05) (n=10).
C) shows the graphical representation of litter size of wild type (n=4) matings and shRNA knock down model matings.

FIG. 9: shows the testicular histology of ten weeks old shRNA knock down model showing varying degree of abnormalities in seminiferous tubules (20× magnification)
A) Seminiferous tubules of ISG12 model with thickened basement membrane.
B) Seminiferous tubules of TGM2 model showing tubular vacuolization.

### SUMMARY OF THE INVENTION

The present invention is towards a rapid process for producing knock down models using shRNA in mammals. The present invention also involves making permanent lines of shRNA knock down models produced by the method. A novel, non surgical, user friendly and relatively inexpensive rapid method has been invented for making permanent lines of shRNA knock down models of non human vertebrates. A specific gene is knocked down by electroporating shRNA construct into the testis of a non human vertebrate. The invention also relates to the incorporation of genes into the germline of non human vertebrate so that it could be inherited. The method is the easiest and fastest in comparison to all other existing methods.

### DESCRIPTION OF THE INVENTION

The present invention provides a rapid process for generating knock down non human vertebrate models by electroporation of shRNA construct into the testis. The present invention provides a non-surgical, user friendly rapid method for the generation of permanent lines of shRNA knock down non human vertebrates. This invention is ethically superior as it does not involve any loss of animal life and drastically minimizes the production time and use of animals. Current techniques for making knockout models are cumbersome, require trained personnel, costly infrastructure and require hundreds of eggs collected after killing several females. In contrast, this method neither involves any costly infrastructure nor requires trained personnel. The invention also relates to the quick incorporation of shRNA gene construct into the germline of a species so that shRNA is inheritable. In the present invention various knock down models were generated from different shRNA of various genes comprising Interferon stimulated gene 12, Transglutaminase 2, Nuclear protein 1 and Glioma tumour suppressor candidate region gene 2. The present invention also generates in a single process a variety of knock down models differentially expressing gene specific shRNA, depending on differential shRNA gene incorporation in native genome, so that the choice of the extent of gene knock down also becomes available in a single process.

Prior to setting forth the invention in detail; it may be helpful in understanding there of to define the following terms.

Non Surgical—Any procedure that does not involve surgery.

Testes—The male sex gland in the scrotum in which sperm and testosterone are produced. There is a pair of testes behind the penis in a pouch of skin called the scrotum. The testes make and store sperm, and make the male hormone testosterone.

Non-Human Vertebrate—All vertebrates except human beings like guinea pig, rabbit, rat, dog etc.

DNA—Deoxyribonucleic acid (DNA) it constitutes the primary genetic material of all cellular material, it occurs predominantly in the nucleus.

Spermatogenesis—This process includes all of the nuclear and cytoplasmic changes that transform the primordial germ cells of the male germ line into mature spermatocytes. The formation of mature sperm in the male testes after the onset of puberty.

Seminiferous Tubules—Seminiferous tubules are located in the testes, and are the specific location of meiosis, and the subsequent creation of gametes, namely spermatozoa. This is the tubules lining in the testes that produce sperm.

Genes—A length of DNA that carries the genetic information necessary for production of a protein. Genes are located on chromosomes and are the basic units of heredity.

Promoter—It is a controlling element in the expression of the gene. It serves as a recognition signal for an RNA polymerase and marks the site of initiation of transcription. A promoter is a region of DNA that facilitates the transcription of a particular gene.

RNAi—RNA interference (RNAi), a method for preventing the synthesis of specific proteins in cells by blocking the corresponding genes. It is a mechanism that inhibits gene expression by causing the degradation of specific RNA molecules or hindering the transcription of specific genes. It is a technique for gene silencing. RNA is introduced into the cell to disrupt messenger RNA and prevent it from being translated into a protein. It is a natural cellular process where small interfering RNAs or microRNAs are used to control the normal expression of genes.

Knock out—The excision or inactivation or deletion of a gene within an intact organism or even animal model usually carried out by a method involving homologous recombination.

Knock down—Suppression of the expression of a gene product, typically achieved by the use of antisense oligo deoxynucleotides and RNAi that specifically target the RNA product of the gene. Gene knock down refers to techniques by which the expression of one or more of an organism's genes is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knock down organism”.

shRNA—Small hairpin (shRNA) contains sense and antisense sequences from a target gene connected by a loop, and is expressed in mammalian cells from a vector by a pol III-type promoter. The shRNA is transported from the nucleus into the cytoplasm, where Dicer processes it. Small hairpin RNA is expressed from a DNA template and processed into small RNAs to guide RNAi-mediated targeted mRNA degradation.

shRNA vector—Small hairpin RNA (shRNA) vector is a DNA molecule used as a vehicle to transfer foreign genetic material into another cell. ShRNA vector can express shRNA with the help of its promoter.

Wild type—the normal, typical phenotype of any mammal before genetic mutation takes place. Wild type (or wild type) refers to the phenotype of the typical form of a species as it occurs in nature.

Electroporation—The application of electric current to a living surface (as the skin or plasma membrane of a cell) in order to open pores or channels in cells or tissue through which a biological material (a drug or DNA) may pass. It is the use of electrical pulses to enable cells to take up DNA.

Transgenic animal—A transgenic animal is one that carries a foreign gene that has been deliberately inserted into its genome. The foreign gene is constructed using recombinant DNA methodology.

Transgenic lines—generations of offspring produced by mating of transgenic models.

F1 Generation—F1 Generation is produced by mating of shRNA knock down male with the wild type female.

F2 Generation—Produced by mating of F1 generation siblings positive for transgene integration.

F3 Generation—Produced by mating of F2 generation siblings positive for transgene integration.

Primer—A primer is a strand of nucleic acid that serves as a starting point for DNA replication.

Real time PCR—real-time polymerase chain reaction, also called quantitative real time polymerase chain reaction, is a laboratory technique based on the PCR, which is used to amplify and simultaneously quantify a targeted DNA molecule. It enables both detection and quantification (as absolute number of copies or relative amount when normalized to DNA input or additional normalizing genes) of one or more specific sequences in a DNA sample.

Antibody—Antibodies also known as immunoglobulins are gamma globulin proteins that are found in blood or other bodily fluids of vertebrates, and are used by the immune system to identify and neutralize foreign objects, such as bacteria and viruses. Molecules produced by B cells in response to specific proteins (antigens) carried by infected cells.

The invention is based in part on the realization that current techniques for making knock out models are cumbersome, requires trained personnel and costly infrastructure. These techniques require hundreds of eggs which are collected after killing several females which are injected with hormones. This invention neither requires assisted reproductive techniques nor sophisticated laboratory setup. It does not require highly trained personnel. This novel testicular shRNA knock down technique contributes to developing an ethically superior (deathless) and easily adaptable time saving procedure.

A preferred embodiment of the invention provides a rapid method of generating gene knock down model by inhibiting endogenous gene expression using shRNA comprising the steps of
a. constructing a shRNA knock down construct with a desired shRNA sequence against a target gene as described herewith,
b. DNA electroporation of the linearized shRNA construct of step a) into the testis of non human vertebrate,
c. allowing the model of step b) to sire with non electroporated models,
d. obtaining knock down models from electroporated models, and
e. generating permanent lines of models where the desired gene is knocked down.

In still another embodiment of the invention the desired shRNA sequence is incorporated into vector.

In further embodiment of the invention relates to a vector is short hairpin RNA (shRNA) vector.

Another embodiment of the invention provides an expression vector comprising a short hairpin construct under the control of ubiquitous CMV promoter.

In yet another embodiment of the invention relates to a non human vertebrate model.

In further embodiment of the invention provides a gene expression that is reduced up to but not limited to 90 percent as compared to the wild type.

In still further embodiment of the invention relates to the transgenic knock down model generated whose genome comprises a shRNA sequence silencing gene, which is selected from a group comprising ISG12, TGM2, NUPR1, GLTSCR2.

Another preferred embodiment of the invention relates to a process comprising the following steps:
1. Gene specific shRNA sequences were synthesized and cloned into pRNAT-CMV3.1/Neo vector (GenScript USA Inc.). Positive clones were confirmed by sequencing. ShRNA clones were linearized with Sal I and 4 Kb DNA fragment was eluted.
2. Rapid generation of shRNA knock down models by electroporation of linearized DNA into testis.
3. Integrated gene may be detected by several means well known to those skilled in the art. Non limiting examples include PCR where DNA sample may be analyzed by PCR for expression of gene.
4. Other method of detection used is quantitative Real Time PCR that may be done to investigate the relative presence of the transcribed RNA from the target gene as a result of shRNA knocking down transcribed mRNA. This is also a measure of its efficacy.
5. Other non limiting illustrative examples of detection include Western blot analysis that can be done using an antibody against the protein encoded by the gene. These methods may be employed as alternative or additional methods for evaluating the presence of the gene.
6. In case the target gene(s) plays a role in spermatogenesis, the reproductive status of the shRNA knock down model may be assessed by taking testis weight, sperm count and fertility test.
7. Testicular tissue may also be analyzed directly, for example, by preparing tissue sections.

In some embodiments, it may be preferable to fix the tissue immersing in Bouin's solution. Tissue section can be paraffin embedded. Slides of the tissue may be used for immunohistochemistry or histological staining with eosin and hematoxylin.

In another embodiment, variety of testicular genes—Interferon stimulated gene 12 (ISG12), Transglutaminase 2 (TGM2), Nuclear protein 1 (NUPR1) and Glioma tumour suppressor candidate region gene 2 (GLTSCR2) shRNA knock down models were made to study their effect on spermatogenesis using the method, wherein shRNA sequences specific for these genes were inserted into the vector and permanent lines of shRNA knock down models were produced.

TABLE 1
The advantages of the present invention in comparison to the prior art

| Prior Art | Advantages of the Present Invention |
|---|---|
| Available methods are surgical. One has to cut | It is non invasive and non surgical. The method |
| open the mammal. | is safe. |
| Large numbers of mammals are killed to | It is deathless. No loss of life is required. |
| generate one model. | |
| Available methods are complex, laborious and | It is easiest and fastest. |
| time consuming. | |
| They require costly infrastructure and are very | It does not require any costly infrastructure. |
| expensive. These are very difficult to adopt. | It is very easy to adopt. |
| Most of the available techniques require highly | The method is very simple and it does not |
| trained personnel. | require any trained personnel. |
| Germ line transmission is a major problem and | The gene is integrated directly in the germ line |
| is a matter of chance. | cells and transferred to the next generation. |
| Expression of the gene is transient. | Expression of the gene is stable. |
| All methods have very low efficiency (0.5%- | It is highly efficient and reproducible method. |
| 10%). | Efficiency of the procedure is 100%. |
| A single event of gene introduction into | Since integration is targeted to testicular |
| oocytes results at the most generation of one | germinal stem cells, a single event of gene |
| transgenic model. | introduction into testis can result into |
| | generation of several transgenic models upon |
| | mating. |
| Gene introduction in 100 oocytes results in the | Gene introduction in the testes of 10 mammals |
| generation of approximate 8 transgenic | makes all of them capable to sire transgenic |
| mammals (8% transgenic efficiency-Our lab | offsprings (100% transgenic efficiency). |
| oocyte injection data) and 0.5 to 10% | |
| transgenic efficiency (Hirabayashi et al., | |
| 2001). | |

### EXAMPLES

The examples are described for the purposes of illustration and are not intended to limit the scope of the invention. Although specific embodiments of the present invention will now be described with reference to the drawings, it should be understood that such embodiments are by way of example only and merely illustrative of but a small number of many possible embodiments which can represent applications of the principles of the present invention. During the trial, various genes were experimented and a few are illustrated here below.

Genes detected in the testis, but whose function in testis are not known were selected from the testis—Interferon stimulated gene 12 (ISG12), Transglutaminase 2 (TGM2), Nuclear protein 1 (NUPR1) and Glioma tumour suppressor candidate region gene 2 (GLTSCR2). The roles of these genes in the testes are not known so far. The shRNA constructs are designed with the aim of knocking down a specific gene expression. The shRNA sequences specific to the genes were synthesized and cloned into a suitable vector, preferably pRNAT-CMV3.1/Neo vector and positive clones were later confirmed by sequencing. The scope of present invention is not limited only to the four various kind of gene examples described here, since it is possible to carry out this exercise with all types of genes.

### Example 1

### ISG12 (Interferon Stimulated Gene 12)

Interferons exert their biological function mainly through the activation of interferon-stimulated genes (ISGs). ISG12 belongs to a family of small, interferon α inducible genes. The interferon inducible gene 12 (ISG12) located at the nuclear envelope is upregulated by interferons from immune cells. ISG12 is known to be involved in allergies. ISG12 might therefore represent a target for novel therapeutic strategies for the treatment of vascular diseases.

### Example 2

### TGM2 (Transglutaminase 2)

TGM2 belongs to the family of transglutaminase. Like other transglutaminases, it crosslinks proteins between an ε-amino group of a lysine residue and a γ-carboxamide group of glutamine residue, creating an inter- or intramolecular bond that is highly resistant to proteolysis (protein degradation). It is particularly notable for being the autoantigen in coeliac disease, but is also known to play a role in apoptosis, cellular differentiation and matrix stabilisation.

### Example 3

Construction of shRNA Knock Down Construct

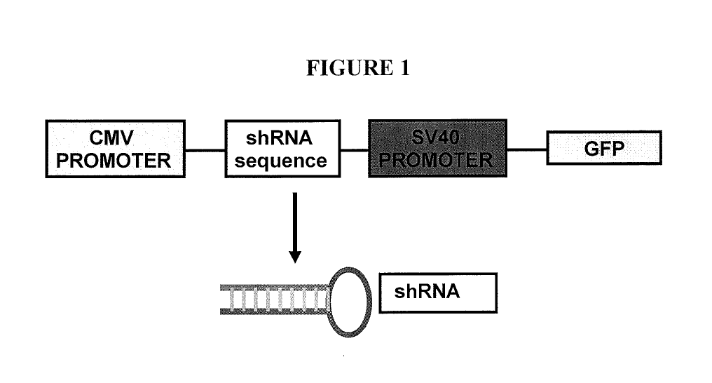

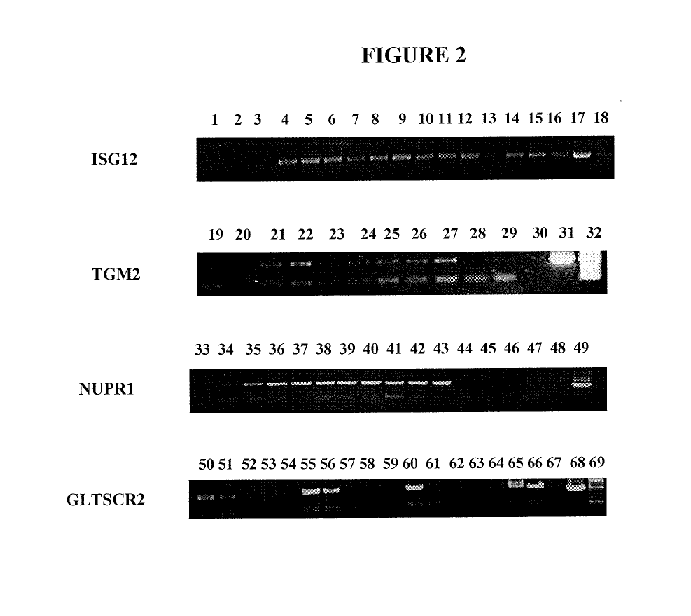

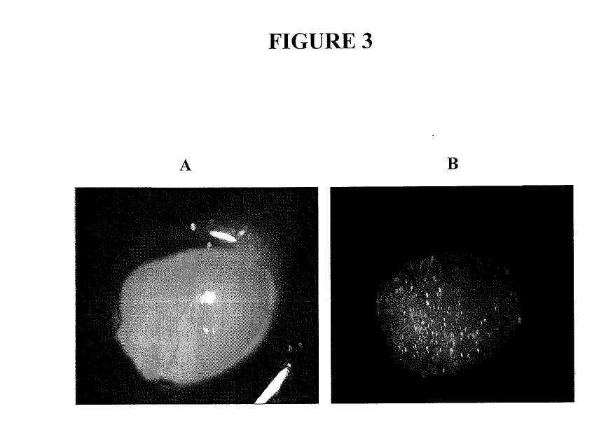

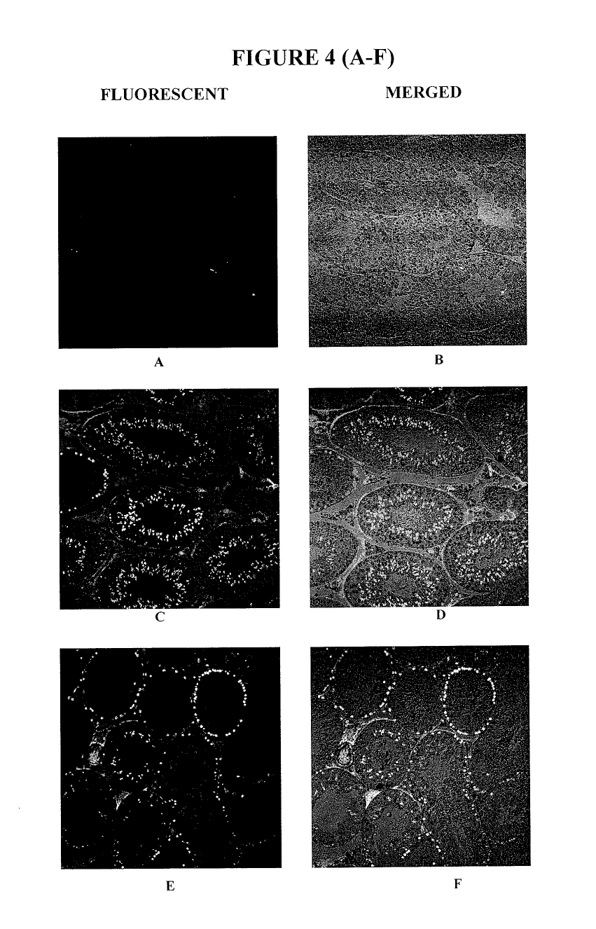

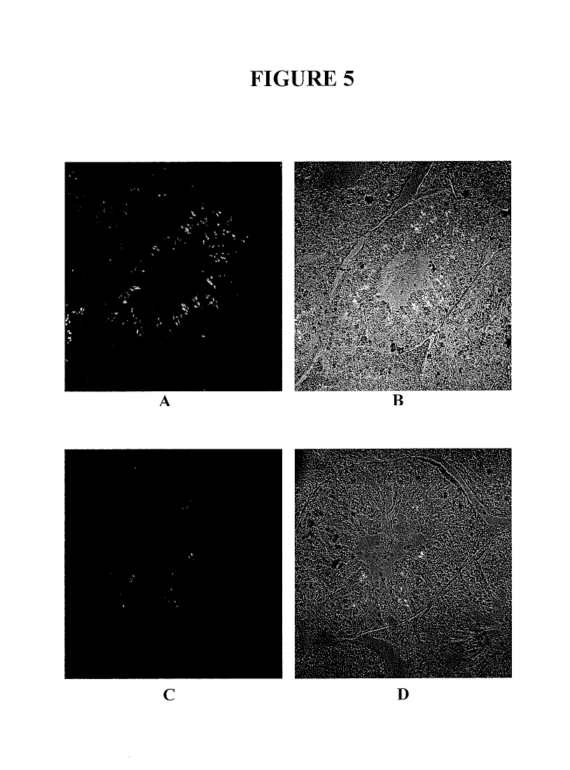

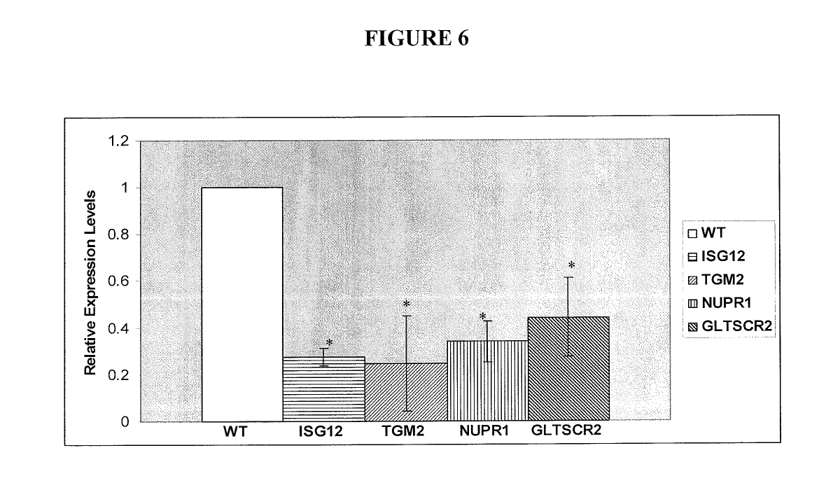

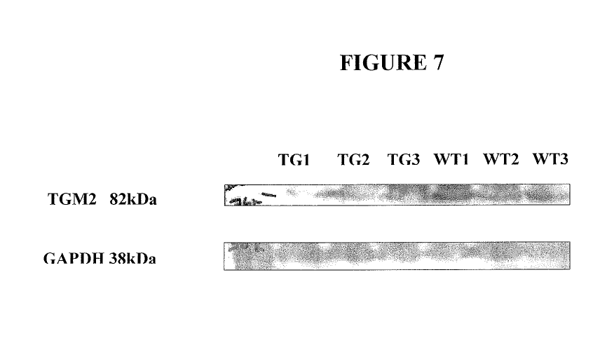

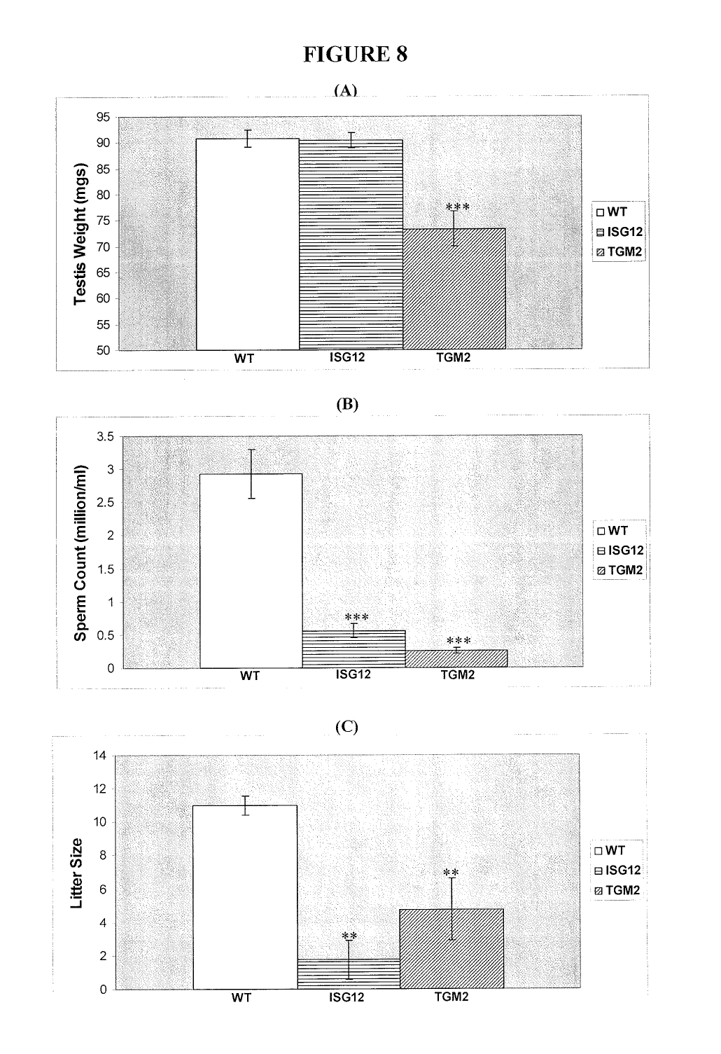

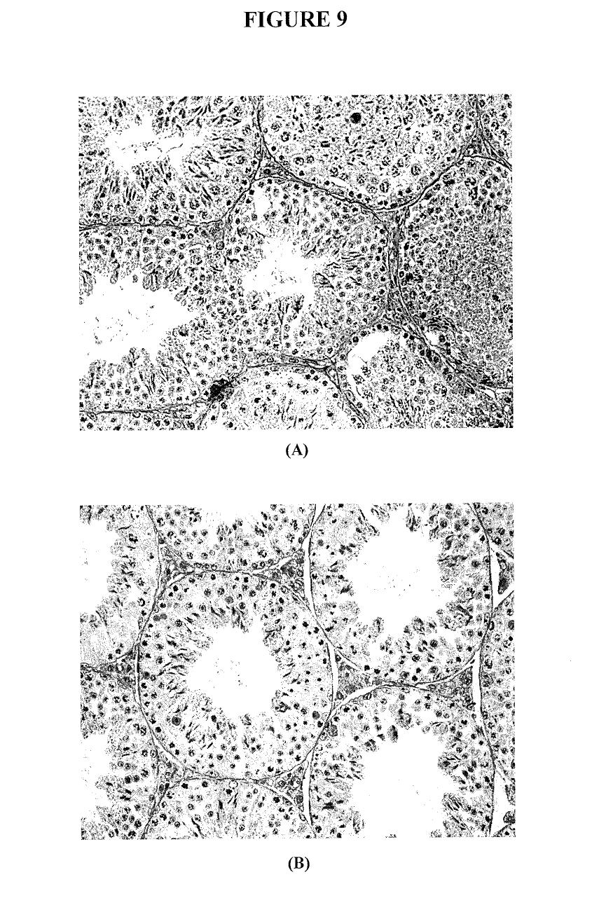

The shRNA constructs can be designed with the aim of knocking down specific gene expression (FIG. 1). Gene specific shRNA sequences may be synthesized and cloned into pRNAT-CMV3.1/Neo vector (GenScript USA Inc.). The shRNA vector comprises of CMV promoter which drives the expression of shRNA and a SV40 promoter drives the expression of the GFP. This vector carries GFP for convenient tracking. Gene specific ShRNA cassettes can be easily inserted into the vectors between BamHI and AfIII sites. Positive clones may be confirmed by sequencing. shRNA clones can be linearized with Sal 1 and 4 Kb DNA fragment is eluted. Suitable shRNA sequences for the knock down of a given target gene are mentioned in Table 2.

TABLE 2
shRNA sequences for the knocking down of all the four genes.

| GENE | FORWARD OLIGO | REVERSE OLIGO |
|---|---|---|
| ISG12 | GATCGTACCAATTGGAGCTTAGGAGATGACACTTCTATTCAA | TTAAGTACCAATTGAAAAAAAGCTTAGGAGATGACACTTCTA |
| | GAGATAGAAGTGTCATCTCCTAAGCTTTTTTTCAATTGGTAC | TCTCTTGAATAGAAGTGTCATCTCCTAAGCTCCAATTGGTAC |
| TGM2 | GATCGTACCAATTGGTCTGTCAAGTTCATCAAGAGTGTTCAA | TTAAGTACCAATTGAAAAAATCTGTCAAGTTCATCAAGAGTG |
| | GAGACACTCTTGATGAACTTGACAGATTTTTTCAATTGGTAC | TCTCTTGAACACTCTTGATGAACTTGACAGACCAATTGGTAC |
| NUPR1 | GATCGTACCAATTGGAACCTAGAGGATGAAGATGGAATTCA | TTAAGTACCAATTGAAAAAAAACCTAGAGGATGAAGATGGA |
| | AGAGATTCCATCTTCATCCTCTAGGTTTTTTTTCAATTGGTAC | ATCTCTTGAATTCCATCTTCATCCTCTAGGTTCCAATTGGTAC |
| GLTSCR2 | GATCGTACCAATTGGCCTTGAGAATCATTCTAAGATCCTCAA | TTAAGTACCAATTGAAAAAACCTTGAGAATCATTCTAAGATC |
| | GAGGGATCTTAGAATGATTCTCAAGGTTTTTTCAATTGGTAC | CCTCTTGAGGATCTTAGAATGATTCTCAAGGCCAATTGGTAC |

### Example 4

### Non Invasive External Electroporation of the Testis

For the purpose of DNA electroporation, linearized DNA fragment are eluted. A non human vertebrate male may be used for DNA electroporation. Non human vertebrates were anesthetized by intra-peritoneal injection of a mixture of ketamine hydrochloride (45 mg/kg) and xylazine hydrochloride (8 mg/kg). Hair can be removed from lower abdominal and scrotal area and the area was cleaned using savlon followed by betadiene. Testis can be pushed into scrotal sacs by pressing the lower abdominal region and held externally. Solution of DNA is optionally mixed with Trypan blue (0.04%) to monitor the accuracy of the injection. DNA was loaded into a Hamilton syringe. Few microlitres (10 ul of 0.2 μg/μl in case of mice) of linearized vector (containing DNA for the production of shRNA knock down model) reconstituted in autoclaved water is injected at each of the three injection sites (Upper/mid/lower portion of the testis). DNA is injected using needle of Hamilton syringe into both the testis of anesthetized mammal. A time lapse of 30 seconds before pulling out the needle prevented the back flow of the DNA solution. The testis turns bluish after DNA injection. Electroporation is generally done by holding testes externally along with scrotal sacs between a pair of sterile tweezer type electrodes (Harvard Apparatus, Inc. USA) using 50 to 60V eight square wave electric pulses in alternating direction (changing pole of electrode after four pulses) with a time constant of 0.05 second and an inter-pulse interval of ˜1 second via an electric pulse generator (Electroporator ECM2001, BTX Instrument Division, Harvard Apparatus, Inc., USA). The whole procedure is accomplished within 10 minutes. Two males per shRNA construct may be electroporated.

### Example 5

### Propagation of the Transgene

Once a cycle of spermatogenesis is completed (about 35 days in case of mice, this may vary in other mammals) post electroporation, each electroporated male is cohabitated with two wild type females for natural mating. This ensured that electroporated spermatogonial stem cell becomes sperm (35 days in case of mice, this may vary in other mammals) and fertilize egg to generate transgenic offspring. Offspring born are analyzed for transgene integration by normal PCR. Real Time PCR is used to assess the efficacy of knock down and those positive for the transgene are considered as F1 generation. F2 generation can be produced by breeding using positive male and female shRNA knock down non human vertebrates from F1 generation (siblings) which may be cohabitated for 3 weeks. Offspring born can be again analyzed to check whether the transgene is stably integrated and propagated to consecutive generation.

### Example 6

Isolation of Genomic DNA (gDNA) and Polymerase Chain Reaction

A tissue from offspring sired by electroporated males are taken and lysed for 16 hours at 55° C. in high salt digestion buffer containing 50 mM Tris HCl, 1% SDS, 100 mM NaCl, 100 mM EDTA and 1200 μg/ml Proteinase K. The lysate can be processed for isolation of DNA using phenol-chloroform extraction followed by ethanol precipitation. Extracted genomic DNA is subjected to PCR analysis using primers. Primers are designed with Forward primer on SV40 promoter and reverse primer on GFP gene, so that all knock down models generated against various gene specific shRNA may be screened using same set of primers (Table 3). Every PCR reaction set has two controls. PCR of plasmid is used as a positive control, and PCR of gDNA obtained from wild type is used as a negative control. The PCR reaction is performed using Perkin Elmer Thermal Cycler. Reaction conditions are as follows: 94° C. for 5 minutes followed by 30 cycles of 94° C. for 30 seconds, 60° C. for 30 seconds and 72° C. for 30 seconds. The gDNA of offspring is screened for the presence of the gene by PCR. Presence of a 500 bp product in PCR confirmed genomic integration of the constructs in shRNA knock down models (FIG. 2).

TABLE 3
Primers used for genomic PCR analysis

| | Primer sequences |
|---|---|
| | 5′→3′ |
| Forward | GCCCCATGGCTGACTAATTT |
| Reverse | GTATCGCCCTCGAACTTCAC |

PCR Analysis of F1, F2 and F3 generation of shRNA knock down models for transgene expression was done and presence of a 500 bp band confirmed that the gene has integrated. (Table 4, 5 and 6)

TABLE 4
Analysis of F1 generation

| | | Offspring positive for transgenic/Total | |
|---|---|---|---|
| | | no. of offspring born | |
| | Electroporated | On mating with | On mating with |
| Gene | male number | wild type ♀1 | wild type ♀2 |
| ISG12 | 7I | 11/12 | No offspring born |
| | 8I | 16/16 | No offspring born |
| TGM2 | 5T | 2/6 | 7/7 |
| | 6T | 8/12 | 6/6 |

TABLE 5
Analysis of F2 generation

| | F1 siblings identity no. | Offspring positive for transgenic/ |
|---|---|---|
| Gene | (♂no. × ♀no.) | Total no. of offspring born |
| ISG12 | 9 × 10 | No offspring were born |
| | 10 × 11 | No offspring were born |
| | 7 × 12 | 2/2 |
| | 8 × 3 | No offspring were born |
| TGM2 | 1 × 7 | 4/5 |
| | 1 × 8 | 3/5 |
| | 10 × 7 | 0/3 |
| | 11 × 9 | No offspring were born |

TABLE 6
Analysis of F3 generation

| | F1 siblings identity no. | Offspring positive for transgenic/ |
|---|---|---|
| Gene | (♂no. × ♀no.) | Total no. of offspring born |
| ISG12 | Line did not progressed after F2 generation | |
| TGM2 | 18 × 20 | 2/8 |
| | 21 × 24 | 1/5 |

### Example 7

### Green Fluorescent Protein (GFP) Expression in Whole Testes

Whole testes of ten weeks old shRNA knock down models were checked for GFP expression under UV light stereo-zoom microscope SMZ-1500 (Nikon) fitted with epi-fluorescence attachment. Photography was done with DS-5M camera assisted with Digital sight DS-LI software. Presence of GFP which was a marker in our construct enabled us to assess integration of our shRNA construct. GFP expression was seen in the testis of knock down model while wild type showed no expression of GFP (FIG. 3).

### Example 8

Immunolocalization of GFP and TGM2 in Wild Type and shRNA Knock Down Model

Immunohistochemistry was performed using Bouin's fixed and paraffin-embedded testicular sections of wild type and F1 generation of shRNA knock down model. Testicular sections are deparaffinized, rehydrated by successive series of ethanol and rinsed in distilled water. Antigen retrieval are done and the samples blocked using goat serum, permeabilized, and then incubated with 1:250 dilution of a primary antibody (rabbit anti mouse polyclonal anti-TGM2 antibody or mouse monoclonal anti-GFP antibody) for overnight at 4° C. Later, after PBS washings, sections are incubated with 1:250 dilution of anti-mouse or anti-rabbit Alexa Fluor 488 (green)-labeled secondary antibody for 4 hours at room temperature in dark. Sections are analyzed for fluorescence using Olympus IX81 microscope equipped with fluo view SV1000. A specific staining of GFP was observed in the testes of shRNA knock down models of ISG12 (FIGS. 4C and 4D) as well as TGM2 (FIGS. 4E and 4F) confirming integration of the transgene. Wild type (FIGS. 4A and 4B) showed no expression of GFP. TGM2 expression was higher in wild type (FIGS. 5A and 5B) and diminished in TGM2 shRNA knock down model (FIGS. 5C and 5D).

### Example 9

### Quantitative Real Time PCR Analyses of Gene Knock Down

To confirm gene knock down and measure its efficacy, the relative expression of gene knock down in testis of all the four shRNA knock down models of ISG12, TGM2, NUPR1, GLTSCR2 may be investigated by quantitative Real Time PCR. Testes of shRNA knock down models can be snap-frozen, tissue can be ground in pestle-mortar and stored in Trizol (Sigma chemical Co., USA) at −80° C. RNA may be isolated from Trizol treated samples using manufacturer's instructions. Real time PCR can be done using different primers specific for respective genes (Table 7). 0.5 μg of RNA was treated with Dnase I (0.5 μg) for 15 min at 25° C. Reaction may be terminated by adding 1 μl of 25 mM EDTA and incubating at 65° C. for 10 min. RNA can be reverse transcribed using Promega kit following manufacturer's instructions. Real time PCR may be performed using 1 μl of cDNA in a reaction volume of 10 μl. SYBR green used from Applied Biosystem.

TABLE 7
Primers used for Real time PCR analysis
of gene expression

| | Forward Primer | Reverse Primer |
|---|---|---|
| cDNA | Sequence 5′ to 3′ | Sequence 5′ to 3′ |
| NUPR1 | AGCAGGACCTAGGCCTGCT | CTTCTTGCTCCCATCTTGCC |
| GLTSCR2 | AGCCTCCTACAACCCAACCT | CTGTCGCTCCAGCTTTTCTG |
| ISG12 | GCCTGGTAGCCACACTCCAA | AAGCTCAGAGCAAGGCTCCA |
| TGM2 | CGACGGGAATATGTCCTACG | ATTCCATCCTCGAACTGCCC |

As shown in FIG. 6, there was a significant decrease in the mRNA levels of corresponding gene in the respective shRNA knock down model relative to wild type (Expression level of the wild type was taken as unit 1.0). Expression levels of target m-RNA in shRNA knock down models decreased by 73% in ISG12, 76% in TGM2, 66% in NUPR1 mice and 56% in GLTSCR2 with respect to mRNA expression in wild type.

### Example 10

Protein Extraction and Western Blot Analysis of TGM2 Protein Levels in Wild Type and shRNA Knock Down Model

Testes samples from three different wild type and three different TGM2 shRNA knockdown models may be lysed with ice-cold PBS (pH 7.4) containing 50 mM Tris Chloride, 150 mM NaCl, 1% Triton X100 and protease inhibitors (1 mM PMSF, 1 μg/ml aprotinin and 1 μg/ml leupeptin) and lysates may be centrifuged (13,000×g, 4° C., 30 min). 20 μg protein from supernatant may be resolved by SDS-PAGE and transferred to a nitrocellulose membrane. After blocking, the membranes may be incubated with rabbit anti mouse polyclonal transglutaminase2 antibody, 1:500 (Abcam, USA cat. no. Ab421) at 4° C. for overnight and then with the goat anti-rabbit horseradish peroxidase-conjugated secondary antibody (1:5000) for 2 hours at room temperature. The blots can be developed using chemiluminescence agent (Amersham Biosciences). Testicular samples from different TGM2 shRNA knock down models and wild type showed 82 kDa TGM2 protein. However, the expression level of proteins were significantly lower in shRNA knock down models as compared to wild type. GAPDH was also assessed by western blotting as loading control (FIG. 7).

### Example 11

### Testis Weights, Sperm Counts and Fertility Assessment

Since the genes belonged to testicular cells, testes weight of wild type and shRNA knock down males at ten weeks of age may be recorded. At the age of ten weeks, testes of both wild type and shRNA knock down model were weighed. Because of the expressing shRNA, testicular weights of TGM2 were significantly decreased (73.3±3.32 mg) as compared to wild type (90.8±1.64 mg). However, testicular weight of ISG12 was found to be 90.4±1.5 mg (FIG. 8A).

To evaluate the biological relevance of the described defects of the gene on sperm production and reproductive capability of knock down model, the concentration and motility of epididymal spermatozoa and the litter size of productive matings were analyzed. Total numbers of sperms present in cauda epididymus can be counted after releasing the sperms in 1 ml of phosphate-buffered saline by puncturing the epididymus at several sites. The total sperm count may be assessed by using a hemocytometer. We observed that spermatozoa number decreased significantly (P<0.05) in epididymal semen of shRNA knock down model as compared to wild type. Sperm counts observed were 0.56±0.10×10⁶/mL/epididymus in ISG12 and 0.26±0.04×10⁶/mL in TGM2 model. In comparison, the sperm counts observed from wild type were 2.93±0.37×10⁶/mL (FIG. 8 B).

Male and female ShRNA knock down model from F1 generation (siblings) may be cohabitated for few weeks. This ensured exposure of female to the male at least through five ovarian cycles. Litter size can be determined as a mean to assess fertility of parent. Similarly, Wild type were also assessed for fertility. To evaluate fertility in shRNA knock down models, three positive males from F1 generation were cohabitated with three positive female siblings for few weeks. The total numbers of offspring born from productive matings were recorded. The average litter size was 1.75±1.18 in ISG12 and 4.75±1.84 in TGM2 model which is significantly lower (P<0.05) in comparison to wild type where the average litter size was 11.0±0.5 (FIG. 8 C). This may authenticate the biological effect of knocking down through our method.

### Example 12

### Histological Examinations

The left testes from shRNA knock down models and wild type may be fixed by immersion in Bouin's solution at room temperature for 24 hours. Testes may be dehydrated through an ethanol series, embedded in paraffin wax, sectioned by standard procedures and sections of 4 μm were obtained and stained with hematoxylin and eosin for evaluating the status of spermatogenesis. Stained slides may be examined using bright field microscopy. To examine the consistency of the phenotype, testes from several mammals of each knock down line may be examined. There were several significant histological differences in the testes of wild type and shRNA knock down models. The regressed testes of the shRNA knock down model showed atrophic seminiferous tubules as compared to wild type, with a range of abnormalities varying in severity between the tubules. The tubules decreased in outer diameter as the atrophy became prominent. Presence of multiple vacuoles with disorganized seminiferous epithelium, presence of multinucleated giant cells and the absence/severe depletion of sperms were some of the common features seen in the seminiferous tubules of all these testes. Apart from these abnormalities, there were some specific features associated with respective genes. The basement membrane around the seminiferous tubules of ISG12 shRNA knock down model was thicker than the seminiferous tubules around the wild type (FIG. 9A). Large numbers of multiple vacuoles of varied diameter were noticed in the testicular tubules of TGM2 model (FIG. 9B). Apart from all these features, certain normal seminiferous tubules were also seen in all the shRNA knock down model, however their percentage was very low.

### Example 13

### NUPR1 (Nuclear Protein 1)

NUPR1 promotes cellular growth and participate in the response to proapoptotic stimuli in a way that helps the tissue counteract diverse injuries. NUPR1 may contribute to the metastatic phenotype. It is seen to be overexpressed in prostate tumors. It is required for leutinizing hormone β promoter activity and its gene expression. It is expressed in variety of tissues including liver, pancreas, testis, ovary (Million Passe et al., 2008). The shRNA knock down model were generated using the same method and were confirmed by PCR and Real time PCR (FIG. 2 and FIG. 6). Presence of 500 bp product confirmed the integration of the shRNA construct. Details of Real time PCR are mentioned in example 9. The other results were also similar to ISG 12 and TGM2 knock down models.

### Example 14

### GLTSCR2 (Glioma Tumor Suppressor Candidate Region Gene 2)

GLTSCR2 plays a role in cell proliferation and apoptosis. Recent studies show aberrations in GLTSCR2 expression in glioblastoma and neuroblastoma. GLTSCR2 takes part in the suppression of tumor growth and development. GLTSCR2 binds to PTEN tumor suppressor and regulates its stability in cells. The shRNA knock down model were generated using the same method and were confirmed by PCR and Real time PCR (FIG. 2 and FIG. 6). Presence of 500 bp product confirmed the integration of the shRNA construct. Details of Real time PCR are mentioned in example 9. The other results were also similar to ISG12 and TGM2 knock down models.

TABLE 8
Different shRNA constructs used for the generation of knock down
models are involved in different type of functions.

| Name of the shRNA construct | Probable known function |
|---|---|
| Interferon stimulated gene 12 (ISG12) | Involved in allergies |
| Transglutaminase2 (TGM2) | Involved in apoptosis |
| Nuclear protein 1 (NUPR1) | Involved in metastasis |
| Glioma tumor suppressor candidate | Play a role in the suppression |
| region gene 2 (GLTSCR2) | of tumour growth |

### ADVANTAGES IN INDUSTRIAL APPLICATIONS

Using non surgical and ethically superior method of invention one can reduce the number of mammals used for any study. One can get a desired transgenic line without sacrificing any mammal. This would be a big help in generating gene knock down large animal models which is difficult to do with other existing techniques. It does not require any trained personnel and costly infrastructure so it is economically better method.

The ability to knock down genes stably through germline transmission of permanently silenced genes has some important advantages. Hood et al., 2004 stated that when a gene is considered silenced by mechanism of RNAi, the expression is typically reduced by 70% or more. The present invention allow shRNA mediated knock down to be used in inhibiting the expression of essential genes, which cannot be knocked out completely because of embryo lethality.

ShRNA allows researchers to inactivate the gene and observe in real time the metabolic and cell biological changes or any other phenotypic changes of the cell, and characterize the role of the gene in that particular situation. Genome wide screening using RNAi libraries via this method will help researchers learn more about global questions in systems biology, elucidating the nature and role of the complex, often interrelated pathways and signaling networks at work in organisms.

We claim:

1. A rapid method of generating gene knock down model by inhibiting endogenous gene expression using shRNA comprising the steps of;
a. constructing a shRNA knock down construct with a desired shRNA against target gene as described herewith,
b. DNA electroplating the linearized shRNA construct of step a) into the model,
c. allowing the model of step b) to sire with non electroporated models,
d. obtaining knock down models from electroporated models, and
e. generating permanent lines of models where the desired gene is knocked down.

2. The method as claimed in claim 1, wherein the desired gene inserted can be shRNA construct or siRNA construct consisting of a double stranded DNA or a single-stranded DNA.

3. The method as claimed in claim 1 wherein desired shRNA construct is incorporated into vector.

4. The method as claimed in claim 3, wherein vector is short hairpin RNA (shRNA) vector.

5. The method as claimed in claim 4, wherein said vector is an expression vector comprising a short hairpin construct under the control of ubiquitous CMV promoter.

6. The method as claimed in claim 5, wherein the promoter is tissue or cell type specific.

7. The method as claimed in claim 1, wherein the said model is a non human vertebrate.

8. The method as claimed in claim 1 wherein the gene expression is reduced up to but not limited to 90 percent as compared to the wild type.

9. A method as claimed in claim 1, wherein transgenic knock down model is generated whose genome comprises a shRNA sequence silencing gene, which is selected from a group comprising ISG12, TGM2, NUPR1, GLTSCR2.

10. The method as claimed in claim 9, wherein the knock down models have disrupted spermatogenesis.

11. The method as claimed in claim 1, wherein desired shRNA construct is integrated randomly in the genome of the host.
